(19) United States
(12) Patent Application Publication
(10) Pub. No.: US 2012/0229804 A1
(43) Pub. Date: Sep. 13, 2012

(54) BOROSILICATE GLASSWARE AND SILICA BASED QMA'S IN 18F NUCLEOPHILIC SUBSTITUTION: INFLUENCE OF ALUMINUM, BORON AND SILICON ON THE REACTIVITY OF THE 18F- ION

(75) Inventors: Alan Peter Clarke, Oslo (NO); Ivar Martinsen, Oslo (NO); Anders Svadberg, Oslo (NO); Ole Kristian Hjelstuen, Oslo (NO)

(73) Assignees: Medi-Physics, Inc., Princeton, NJ (US); GE Healthcare Limited, Buckinghamshire (GB)

(21) Appl. No.: 13/510,455

(22) PCT Filed: Dec. 21, 2010
(86) PCT No.: PCT/US10/61411

§ 371 Date: May 17, 2012

Related U.S. Application Data
(60) Provisional application No. 61/288,354, filed on Dec. 21, 2009.

(51) Int. Cl. G01N 33/38; H01J 49/26; G01J 3/443

(52) U.S. Cl. 356/316; 250/282

(57) ABSTRACT

Aluminum, boron and silicon were identified as potential leachables from borosilicate glassware and a silica based QMA during handling of aqueous ¹⁸F. The addition of only 0.4 ppm aluminum as AlCl₃ in the eluent vial resulted in a strong reduction in the labeling yield of a model [¹⁸F]fluoride S_{N}2 reaction (from 80 to 40% incorporation). The addition of boron as KBO₂ and silicon as NaSiO₃ did not result in any significant decrease in labeling yield. Interestingly, there was an interaction effect between AlCl₃ and KBO₂ in which the negative effect from AlCl₃ on labeling yield was counteracted by KBO₂. The present invention demonstrates that aluminum and boron from borosilicate glassware have a strong influence on the labeling yield in nucleophilic S_{N}2 reactions with n.c.a [¹⁸F]fluoride.

### FIELD OF INVENTION

The present invention claims a method for detecting aluminum, boron, or silicon in borosilicate glassware and a silica based anion exchange columns (QMA) during handling of aqueous 18F, wherein the method is analyzed with inductively coupled plasma atomic emission spectroscopy to make a labeled product. Specifically, the present invention also claims a method for detecting aluminum, boron, or silicon in borosilicate glassware and a silica based QMA during handling of aqueous 18F, wherein said method comprises preparing a K222/K2O3 eluent mixture in 80/20 MeCN:H2O and 0.825 ml was stored at room temperature in a 3 ml borosilicate glass vial, capped with a Teflon coated chlorobutyl stopper whereby the content was analyzed with inductively coupled plasma mass spectroscopy to make a labeled product. The present invention further claims a process for detecting aluminum, boron, or silicon in borosilicate glassware and a silica based QMA during handling of aqueous 18F, wherein the method is measured directly by inductively coupled plasma atomic emission spectroscopy to make a labeled product.

Nucleophilic substitution with no-carrier-added (n.c.a.) [¹⁸F]fluoride is the key radiolabeling technique in Positron Emission Tomography (PET). Production of [¹⁸F]fluoride is most commonly preformed by proton irradiation of [¹⁸O]H₂O targets via the ¹⁸O(p,n)¹⁸F reaction and obtained as high yielding aqueous n.c.a. ¹⁸F (Cai et al., 2008; Guillaume et al., 1991; Schlyer, 2003). The subsequent nucleophilic substitution normally takes place in an aprotic but polar environment. In addition, a phase transfer agent such as an aminopolyether or a tetraalkyl ammonium salt is added to enhance the reactivity of the ¹⁸F⁻ ion (Hamacher et al., 1986; Brodack et al., 1988). However, the low mass of ¹⁸F involved makes such a reaction sensitive to contaminants. In addition, the strong tendency of the fluoride ion for complex formation with lewis acids or heavy metals makes the reaction very sensitive and highly variable labeling yields often results (Nickles et al., 1986; Berridge and Tewson, 1986a; Tewson 1989). So far, the demand for “clean” ¹⁸F⁻ has to a large extent been satisfied through upgrading of cyclotrons and targetry systems. Improved cyclotron technology from only a few global vendors have standardized the bombardment process, and continuous development in targetry over the last two decades have greatly improved the impurity profile of aqueous ¹⁸F⁻ at end of bombardment (Kilbourn et al., 1984, 1985; Berridge and Tewson, 1986b; Wieland et al., 1986; Iwata et al., 1987; Solin et al., 1988; Tewson et al., 1988; Schlyer et al., 1993; Berrigde and Kjellström, 1999; Wilson et al., 2008).

Nonetheless, inconsistent yields from ¹⁸F substitution reactions are still typical. Various ionic contaminants, both from the target and the successive handling, are most likely present and are causing reduced yields. There are means of purifying aqueous ¹⁸F⁻, like conversion into the intermediate [¹⁸F]fluorotrimethylsiliane (Hutchins et al., 1985; Rosenthal et al., 1985;) or via electrochemical procedures (Hamacher et al., 2002; Reischl et al., 2002). However, these and other measures are cumbersome and seldom implemented. Most likely, ionic contaminants arise during handling of ¹⁸F after irradiation. Transportation and flow of the aqueous ¹⁸F from target to the reaction chamber always involves several contact surfaces. Plastic tubing, borosilicate glassware and a QMA trapping column are typically in place. The importance of these factors in ¹⁸F labeling can be exemplified by recent work showing how radiolysis of teflon-tubing/components in a typical synthesis setup represented 90-95% of all carrier fluoride being introduced (Berridge et al., 2009).

In the present invention focus is given on how common leachables of borosilicate glass and a silica-based QMA column could render ¹⁸F un-reactive. There is an extensive use of borosilicate glass in conventional ¹⁸F setups, both manual rigs and automated systems. The container for non-irradiated [¹⁸O]H₂O, collection vial for irradiated [¹⁸O]H₂O, reagent vials and reaction vessels are often made of borosilicate glass. Previous papers have reported that reaction vessels of borosilicate glass can bind aqueous ¹⁸F irreversibly (Brodack et al., 1986; Mudrová and Svoboda, 1972). It has been speculated that this phenomenon is due to ¹⁸F binding to constituents of glass like silicon and boron, forming Si—F and B—F bonds (Nickles et al., 1986). In the present invention, it is shown that leachables from borosilicate glassware and silica-based QMA's bind fluoride in such fashion.

### SUMMARY OF THE INVENTION

The present invention claims a method for detecting aluminum, boron, or silicon in borosilicate glassware and a silica based QMA during handling of aqueous 18F, wherein the method is analyzed with inductively coupled plasma atomic emission spectroscopy wherein said method comprises preparing a K222/K2O3 eluent mixture in 80/20 MeCN:H2O and 0.825 ml was stored at room temperature in a 3 ml borosilicate glass vial, capped with a Teflon coated chlorobutyl stopper whereby the content was analyzed with inductively coupled plasma mass spectroscopy. The present invention further claims a process for detecting aluminum, boron, or silicon in borosilicate glassware and a silica based QMA during handling of aqueous 18F, wherein the method is measured directly by inductively coupled plasma atomic emission spectroscopy.

### DETAILED INVENTION

Leachables of borosilicate glassware and silica based anion exchange columns (QMA's) have been found to influence nucleophilic substitution with ¹⁸F. Aluminum, boron and silicon, all constituents of borosilicate glass, were found as water soluble leachables in a typical PET synthesis setup. Relevant ranges of the leachable quantities were studied based on an experimental design, in which species of the three elements were added to the reaction vessel of a S_{N}2 ¹⁸F reaction. Levels of 0.4-2 ppm aluminum as AlCl₃ had a strong negative influence on labeling yield while 4-20 ppm of boron as KBO₂ and 50-250 ppm of silicon as Na₂SiO₃ did not have a significant impact. Interaction effects between the elements were observed, where particularly KBO₂ reduced the negative effect of AlCl₃ on labeling yield. It is concluded that leachables of borosilicate glassware can easily influence nucleophilic substitution with n.c.a. ¹⁸F⁻ and give variable yields.

Normal borosilicate glassware has the following composition: (Pyrex-7740) consists of: 81% SiO₂, 13% B₂O₃, 2% Al₂O₃, 4% Na₂O (Doremus, 1979). In order to achieve leachables, dissolution must take place. Dissolution of borosilicate glass in aqueous environment is well known (Borchert et al., 1989). It is slow at acidic and neutral pH but accelerates at alkaline pH (Bochert et al., 1989; Doremus, 1979). Dissolution of silica (SiO₂) results from cleaving the Si—O—Si bridges with OH⁻. Dissolved SiO₂ has a complex speciation in aqueous solutions. It is highly dependent on pH, concentration, temperature and ageing (Cotton and Wilkinson, 1988a). Above pH 9, it has propensity to polymerize, although monomeric anions like SiO(OH)₃⁻ and SiO₂(OH)₂²⁻ will dominate in diluted alkaline solutions (Alexander et al., 1954; Yang et al., 2008). In regards of fluorosilicates, there is a large range of conceivable complexes, however at pH above 5 these are hydrolyzed and free fluoride results (Urbansky, 2002). Dissolution of boron trioxide (B₂O₃) is expected to behave similarly to silica (Bochert et al., 1989). B₂O₃ reacts with water to give boric acid: B₂O₃→HBO₂ (metaborate)→B(OH)₃ (boric acid). B(OH)₃ is acidic due to interaction with water molecules: B(OH)₃+H₂O→B(OH)₄⁻+H⁺, pKa=9.24. As with silica, there is a large diversity of water soluble anionic species although with increasing pH, B(OH)₄⁻ dominates (Cotton and Wilkinson, 1988b). As with fluorosilicates, fluoroborates are well known in acidic to neutral aqueous solutions. However at pH 6-8, hydrolysis occurs and free fluoride results (Wamser, 1948).

Dissolution of alumina (Al₂O₃) dissolves into aluminum hydroxide (Al(OH)₃) by hydrolysis with OH⁻ in aqueous environment. In water, Al(OH)₃ is insoluble at near neutrality (pH 7-7.5). At acidic pH (pH<6), Al(OH)₃ is converted into several water soluble, cationic species. At alkaline conditions (pH>8.5), Al(OH)₃ is completely converted into the water soluble Al(OH)4⁻. Aluminum-fluoride complexes exist over a large pH range, although at increasing alkalinity hydroxyl ions outcompetes F for Al and pure Al(OH)₄⁻ results. In terms of ¹⁸F, it has been shown that aluminum binds ¹⁸F strongly and decrease its reactivity (Clark and Silvester, 1966; Mudrova and Svoboda, 1972). Others have reported that passing irradiated ¹⁸O-water over an alumina column followed by elution of the ¹⁸F for purification purposes resulted in essential no yield on attempted nucleophilic substitution (Gatley and Shaughnessy, 1982).

The co-presence of silicon, boron and alumium as water soluble species has yet not been evaluated in regards to reactivity of ¹⁸F. In the present invention, leachable levels of Si, B and Al has been investigated in a typical ¹⁸F nucleophilic synthesis set-up. In this invention, three possible sources of contaminants that could influence a ¹⁸F labeling reaction were investigated; (i) Enriched 18-O target water, (ii) a commonly used silica-based QMA (iii) an alkaline K₂₂₂/K₂CO₃ eluent kept in a borosilicate glass vial. The concentration of Si, B and Al in these sources were determined in order to have a baseline value for further experiments in which the levels could be manipulated. The discovered levels found were then simulated by adding species of silica, boron and AlCl₃ to a typical S_{N}2 substitution reaction with ¹⁸F. An experimental design study was executed in order to disclose not only individual effects, but also potential interaction effects between the salts.

One preferred embodiment encompasses a method for detecting aluminum, boron, or silicon in borosilicate glassware and a silica based QMA during handling of aqueous 18F, wherein said method comprises irradiating clear silanized borosilicate glass vials with [18O]H2O then analyzing with inductively coupled plasma atomic emission spectroscopy to make a labeled product such as [18F] RGD peptide or anti-[¹⁸F]FACBC.

Yet another embodiment details a method for detecting aluminum, boron, or silicon in borosilicate glassware and a silica based QMA during handling of aqueous 18F, wherein said method comprises preparing a K222/K2O3 eluent mixture in 80/20 MeCN:H2O and 0.600 to 0.900 ml preferably 0.825 ml was stored at room temperature in a 2-5 ml preferably 3 ml borosilicate glass vial, capped with a Teflon coated chlorobutyl stopper whereby the content was analyzed with inductively coupled plasma mass spectroscopy to make a labeled product such as [18F] RGD peptide or anti-[¹⁸F]FACBC.

Still a further embodiment of the present invention depicts a method for detecting aluminum, boron, or silicon in borosilicate glassware and a silica based QMA during handling of aqueous 18F, wherein said method comprises utilizing Sep-Pak® QMA light cartridges, originally on chloride form was conditioned with 10 ml 0.05 M K₂CO₃, washed with 20 ml WFI, dried under a stream of N₂ and stored at room temperature thereafter 1.5 ml of WFI was forced through and measured directly by inductively coupled plasma atomic emission spectroscopy to make a labeled product such as [18F] RGD peptide or anti-[¹⁸F]FACBC.

Examples of an [18F] RGD peptide can be found in U.S. Pat. No. 7,410,943, US publication No. WO2004/080492, or US Publication No. WO2006/030291.

### EXAMPLES

The invention is further described in the following examples, which is in no way intended to limit the scope of the invention.

The invention is illustrated by way of examples in which the following abbreviations are used:
hr(s): hour(s)
min(s): minute(s)
ml: milliliter
μg: microgram
mg: milligram
ppm: parts per million
AlCl3: aluminum chloride
KBO2: potassium Borooxide
NaSiO3: sodium silicon oxide
RT: room temperature
Me: methyl
C: celcius

2. Materials and Methods

2.1. General

Enriched [¹⁸O]-water 98%, was obtained from Taiyo Nippon Sanso. Acetonitrile, super purity solvent from Romil, was used for eluent solutions in table 4. Acetonitrile 99.8%, prep solv, obtained from Merck (Darmstadt, Germany) was used everywhere else relevant. Kryptofix 2.2.2 and K₂CO₃ (Merck), Na₂SiO₃*9H₂O (Acros Organics), KBO₂*H₂O (Johnson Matthey), and AlCl₃*6H₂O (Fluka) were all used without further work-up. Water for injection (WFI) was prepared by filtration, ion-exchange and quadruple distillation, obtained in-house.

2.2 Production of [¹⁸F]Fluoride

N.c.a. [¹⁸F]fluoride was produced via the ¹⁸O(p,n)¹⁸F nuclear reaction using a GE PETtrace cyclotron (Norwegian Cyclotron Center, Oslo). Production runs of ¹⁸F were performed by dual-beam, 30 μA irradiation of two equal targets for 60 min (16.5 MeV protons). Targets consisted of silver bodies with Havar foils, each filled with 1.6 ml of ≧98% [¹⁸O]H₂O. After irradiation and removal of the irradiated water from the target, 2-3 ml water (Merck, Water for GR analysis) was used to wash out residual activity (2-3 GBq). Further dilution with WFI, gave 100-150 MBq in 1.0-1.5 ml for use in each run.

2.3 Radiochemistry

All radiochemistry experiments were performed on a fully automated on a GE FASTlab® with disposable synthesis cassettes using one pre-built cassette per run. Briefly, the ¹⁸F was trapped on a polymer based anion exchange resin (Macherey-Nagel, Chromafix®-HCO₃, 45 mg). Fluoride was eluted into a 5 ml cyclic polyfine copolymer (COC) vessel using a mixture of 3.6 mg K₂CO₃, 36 mg Kryptofix in 0.7 ml of 78.5% MeCN (aq). This mixture was dried for 13 min in vacuum under a weak stream of nitrogen at 110° C. by azeotropic distillation. Precursor (1-tert-Butoxycarbonylamino-3-trifluoromethanesulfonyloxy-cyclobutanecarboxylic acid ethyl ester, 40 mg) in 1.2 ml of MeCN was added to the dry residue of the [K/222]^{+ 18}F⁻ complex. Labeling was performed at 85° C. for 3 minutes. The reaction solution was quenched with a total of 12 ml WFI at the end of labeling. Radiochemical yields and purities were confirmed by thin layer chromatography (TLC) which was run on pre-coated plates of silica gel (Merck 60F254) using MeCN/MeOH/H₂O/CH₃COOH (20:5:5:1) as mobile phase. The labeled product was not further deprotected to give anti-[¹⁸F]FACBC, since the reaction of interest in this case was the incorporation of ¹⁸F into the organic molecule.

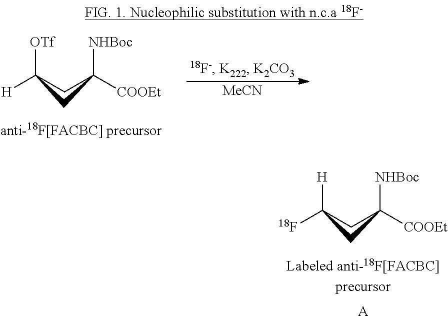

2.4 Screening for Al, B and Si

Concentration of silica, boron and aluminum were determined by either inductively coupled plasma atomic emission spectroscopy (ICP-AES) or inductively coupled plasma mass spectroscopy (ICP-MS). Three possible sources were investigated. (1) Irradiated [¹⁸O]H₂O (3.2 ml) which was kept in clear silanized borosilicate glass vials (Sigma-Aldrich) before analysis with ICP-AES. (2) Sep-Pak® QMA light cartridges from Waters, originally on chloride form was conditioned with 10 ml 0.05 M K₂CO₃, washed with 20 ml WFI, dried under a stream of N₂ and stored at room temperature. A few cartridges were in addition sterilized by gamma-irradiation before storage. After storage, 1.5 ml of WFI was forced through and measured directly by ICP-AES. (3) A K₂₂₂/K₂CO₃ eluent mixture (50 mg/ml K₂₂₂, 10 mg/ml K₂CO₃) in 80/20 MeCN:H₂O, 0.825 ml was stored at room temperature in a 3 ml borosilicate (Fiolax, Sigma-Aldrich) glass vial, capped with a Teflon coated chlorobutyl stopper. The content analyzed with ICP-MS. All ppm values are expressed as μg/ml.

2.5 Statistical Design Setup

To determine the potential interaction effects between the different salts, a full fractional 2-level design with a centre-point was performed (Table 1). The levels of each salt chosen correspond to the amount of pure element. The design was extended by inclusion of single additions of each salt (low and high level). The inorganic salts, AlCl₃, KBO₂ and Na₂SiO₃ were included in the eluent mixture. Each salt was first diluted individually with WFI, then mixed together in the sequence; AlCl₃—KBO₂—Na₂SiO₃. The final eluent solution was mixed in 3 ml borosilicate glass vials (Fiolax, Sigma-Aldrich) in the sequence: K_{222 (MeCN)}, mixture of inorganic salts and then K₂CO₃ (aq). The eluent was made 24 to 72 hours prior to radiosynthesis. The statistical calculations were performed by The Unscrambler software, version 9.6, Camo.

TABLE 1
Full fractional 2-level experimental design overview.

| Entry | Si (ppm) | Al (ppm) | B (ppm) |
|---|---|---|---|
| 1 | 50 | 0.4 | 4 |
| 2 | 250 | 0.4 | 4 |
| 3 | 50 | 2 | 4 |
| 4 | 250 | 2 | 4 |
| 5 | 50 | 0.4 | 20 |
| 6 | 250 | 0.4 | 20 |
| 7 | 50 | 2 | 20 |
| 8 | 250 | 2 | 20 |
| 9 | 150 | 1.2 | 12 |

3. Results and Discussion

3.1 Screening for Leachables

The leachable levels of silicon, boron and aluminum from O-18 enriched target water, Sep-Pak® QMA light and the borosilicate eluent vial, respectively, are summarized in tables 2-4. Measurements of non-irradiated and irradiated O-18 enriched water both showed detectable levels of boron and silicon, while only irradiated samples included levels of aluminum. Generally, the irradiated samples showed higher levels and greater variation of all three elements. Although different irradiation conditions were used in the cited literature and in our own work, the results demonstrate the likely introduction of these elements after irradiation. The borosilicate glass vial used in receiving irradiated water has earlier been considered as a source of boron and silicon (Avila-Rodriguez et al., 2008) and was again found be the case in our work. When the borosilicate vial was replaced with a polypropylene container, the elemental levels dropped considerably and did no longer differ from the non-irradiated samples (Table 2). It is not yet established if the leakage of Al, B and Si from this borosilicate glass follows a mechanism of radiolysis of glass, soluble surface contaminants or a normal dissolution mechanism.

TABLE 2
Measurements of Al, B and Si in irradiated and non-irradiated
O-18 enriched target water.
Results from both literature and own work were included.

| | ICP-MS/AES measurements (ppm) | | | |
|---|---|---|---|---|
| Procedure | Al | B | Si | Reference |
| Non-irradiated | nd | nd | 0.006 | Ehrenkaufer, |
| | | | | 1995 |
| Non-irradiated | <0.0016 | 0.1-0.8 | 0.4-1.6 | Harris et al., |
| | | | | 1989 |
| Irradiation - transfer | 0.04-1.3 | 0.5-4.4 | 0.7-3.8 | Avila- |
| in plastic tubing - | | | | Rodriguez, |
| received in | | | | 2008 |
| borosilicate vial | | | | |
| Irradiation - transfer | <0.02 | 3-11.0 | 8.2-14.0 | This work |
| in plastic tubing - | | | | |
| received in | | | | |
| borosilicate vial | | | | |
| Irradiation - transfer | <0.02 | 0.1 | 0.2 | ″ |
| in plastic tubing - | | | | |
| received in | | | | |
| polypropylene vial | | | | |

nd = not determined.

The commonly used Sep Pak® QMA Light column was investigated as a potential source of water soluble silicon. The combination of a silica-based column with a recommended pH range of 2-9, alkaline pH from a typical K₂CO₃ conditioning solution and its relatively large resin bed-size (130 mg) made the column likely for a silicon contribution. Untreated Sep Pak® QMA Light columns on the Cl⁻ form and 0.05 M K₂CO_{3(aq)} conditioned columns were tested by pushing 1.5 mL WFI through the columns and analyzing the extract by ICP-AES (Table 3). There was a substantial release of silicon from the untreated QMA on the Cl⁻ form. In the K₂CO₃ conditioned form, it was likely that when dried, the actual pH of the stationary phase would exceed the recommended pH range of 2-9. During removal of water, the up-concentration of K₂CO₃ would more likely result in a pH closer to 11.5-12 within the stationary phase. Hence, this increase of alkalinity could result in hydrolysis of the underlying silica. Such drying after conditioning would typically be applied in automated systems that require some storage of a pre-conditioned QMA. Although not dramatic, some silicon was released from a freshly conditioned and dried column. Storage time on the other hand, greatly influenced the levels of released silicon (Table 3). A plateau was reached after 30 days of storage of which there was no increase for the next 120 days. Gamma sterilization of freshly K₂CO₃ conditioned and dried columns did not seem to differ from the non-sterilized samples, as 150 days of storage gave similar results for all conditions. As a descriptor of further development, γ-sterilized columns were followed for another 400 days. The levels of released silicon seemed to be lower at 360 and 600 days vs. 150 days. The overall levels of released aluminum and boron remained low or below the detection limits, as expected.

TABLE 3
ICP determination of Al, B and Si in 1.5 ml WFI extract from
Sep-Pak ® QMA Light.

| | | Storage | | | | |
|---|---|---|---|---|---|---|
| | | time | 1.5 ml H2O extract (ppm) | | | |
| Item | Pretreatment | (days) | pH | Al | B | Si |
| Sep Pak ® | None, Cl⁻ form | 0 | 5.4 | <0.02 | 0.06 | 37.5 |
| QMA light | 0.05M K₂CO₃, | 0 | 7.1 | <0.02 | <0.04 | 5.9 |
| | washed with | 1 | 6.9 | <0.02 | <0.04 | 10.5 |
| | H₂O + dried | 30 | 7.3 | 0.02 | 0.7 | 167 |
| | with N₂ | 150 | 7.1 | <0.01 | 0.8 | 160 |
| | | 150* | 7.3 | 0.02 | 0.7 | 155 |
| | | 360* | 7.8 | 0.09 | 0.7 | 133 |
| | | 600* | 7.1 | <0.01 | 0.8 | 131 |

*γ-sterilized

When a typical K₂₂₂/K₂CO₃ eluent mixture was stored in a borosilicate glass vial, which is highly relevant for automated systems, the levels of all three elements observed in this study increased with storage time. The alkaline pH most likely dissolved the glass material, freeing levels of silicon, boron and aluminum which are all constituents of the borosilicate glass. The dissolution however was rather slow in the beginning. After 7 days, more substantial levels of aluminum was observed (Table 4).

TABLE 4
ICP-MS determination of Al, B and Si in K₂₂₂ eluent
solution stored in borosilicate glass

| | Storage time | Al | B | Si |
|---|---|---|---|---|
| Content | (days) | (ppm) | (ppm) | (ppm) |
| 50 mg/ml K₂₂₂, 10 mg/ml | 0 | <0.02 | 0.1 | 0.2 |
| K₂CO₃ in 80:20 | 1 | <0.02 | 0.1 | 0.3 |
| MeCN:H₂O, 0.825 ml | 7 | 0.2 | 0.3 | 1 |
| | 21 | 0.4 | 6.5 | 9 |
| | 270 | 0.7 | 11.3 | 11 |

Detection limits: Al = 0.02, B = 0.02, Si = 0.06

In total, all three sources investigated; O-18 enriched water, QMA extract and the K₂₂₂/K₂CO₃ eluent all released ppm levels of aluminum, boron and silicon. The findings demonstrate how the content of these three elements may vary in a conventional ¹⁸F nucleophilic set-up. The unknown is how these three elements may interact with the ¹⁸F⁻ ion. It has been speculated that cationic impurities in the target water would not represent a threat towards ¹⁸F when a QMA is applied for ¹⁸F trapping. The rationale being that cations like Al³⁺ would be sent directly through the QMA, while the ¹⁸F is absorbed. (Nishijiama et al., 2002) However, QMA's are normally conditioned with either carbonate or bicarbonate. The alkaline pH would therefore transpose any cationic species of aluminum to either Al(OH)₃₍ₛ₎ or Al(OH)₄⁻₍₁₎. So forth, the QMA would trap any aluminum instead of being non-absorbant. In similar terms, this might apply to Ca²⁺.

Regarding the water soluble silicon found in the pre-conditioned Sep-Pak® QMA, it could be argued that most of it is removed when ¹⁸O—H₂O is flushed through the QMA. However, the possible interaction between species of silicon, boron, aluminum and ¹⁸F combined might form complexes that are not flushed out. In that regard, levels of silicon found was included in the experimental design study in order to investigate its impact on the ¹⁸F labeling reaction. Any ionic leachables evolving from the eluent vial need to pass the QMA in order to reach the reaction vessel. In this study, the added salts of aluminum, boron and silicon were therefore included in the eluent mixture to allow any absorption by the QMA.

The salts AlCl₃, KBO₂ and Na₂SiO₃ were chosen based on their solubility in water and as natural starting points in the plausible speciation in aqueous solutions. AlCl₃ would form free Al³⁺ ions, KBO₂ is an early intermediate when B₂O₃ is dissolved into B(OH)₄⁻ through hydrolysis (Cotton and Wilkinson, 1988b) and Na₂SiO₃ will form SiO(OH)₃⁻ and SiO₂(OH)₂²⁻ when fully hydrated at alkaline pH (Yang et al., 2008).

3.2 Radiolabeling

All radio-TLC performed throughout the study showed only two peaks; free fluoride at the origin and labeled product at the mobile phase front. In the full fractional design, the additions of the three salts had a major impact on labeling yield (Table 4, entries 1-9). The interaction between salts and labeling yield was determined by using partial least squares regression analysis. Since entries 2, 7 and 8 had relatively large standard deviations, the design was extended to include single additions of each salt (Table 4, entries 11-16). With no salts added, consistent labeling yields of 80% was achieved (entry 10, n=4).

TABLE 4
Labeling yield after combined and single additions of silicon as
Na₂SiO₃, boron as KBO₂ and aluminum as AlCl₃ in the eluent vial.

| | | | | Labeling yield |
|---|---|---|---|---|
| Entry | Si (ppm) | B (ppm) | Al (ppm) | (% ± Std) |
| 1 | 50 | 4 | 0.4 | 68 (n = 1) |
| 2 | 250 | 4 | 0.4 | 58 ± 12.5 |
| 3 | 50 | 4 | 2 | 9 ± 0.1 |
| 4 | 250 | 4 | 2 | 6 ± 1.1 |
| 5 | 50 | 20 | 0.4 | 30 ± 2.5 |
| 6 | 250 | 20 | 0.4 | 43 ± 1.6 |
| 7 | 50 | 20 | 2 | 30 ± 8.9 |
| 8 | 250 | 20 | 2 | 31 ± 16.7 |
| 9 | 150 | 12 | 1.2 | 50 ± 0.8 |
| 10 | 0 | 0 | 0 | 80 ± 1.0 |
| 11 | 50 | 0 | 0 | 77 ± 0.9 |
| 12 | 250 | 0 | 0 | 68 ± 6.0 |
| 13 | 0 | 4 | 0 | 59 ± 8.0 |
| 14 | 0 | 20 | 0 | 58 ± 1.0 |
| 15 | 0 | 0 | 0.4 | 40 ± 4.4 |
| 16 | 0 | 0 | 2 | 8 ± 0.4 |

n = varied from 2-4

The regression analysis revealed two significant effects from the tested variables, as illustrated by the bars in FIG. 1. First, there was a strong negative correlation between AlCl₃ and labeling yield. Although this was not surprising, the results illustrate how addition of only 0.4 ppm aluminum as AlCl₃ could pass the QMA and strongly inhibit the labeling reaction. Secondly, there was a clear interaction between the salts, AlCl₃ and KBO₂ (Al*B). The negative impact of AlCl₃ on yield was generally counteracted by KBO₂. It was observed that certain combinations of the two salts proved more beneficial on yield than others. For instance, at aluminum levels of 1-2 ppm, increasing levels of KBO₂ was highly beneficial on yields. A prediction plot, based on the regression analysis, illustrates how yield changed with different levels of AlCl₃ and KBO₂ (FIG. 2). It was speculated that KBO₂ alone made a negative impact on yield, as shown in table 4. However, this negative effect was found insignificant. The addition of Na₂SiO₃ did not show any significant impact on yield.

The effect on yield from each variable is illustrated by each significant regression coefficient. A positive number indicates a positive effect, while a negative number indicates a negative effect from that variable. The significance (p=0.05) of the regression coefficients is given by the uncertainty in the interval on the top of each bar. The response surface is showing how yield (%) varied as a function of different levels of boron as KBO₂ and aluminum as AlCl₃ (ppm).

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

1. A method for detecting aluminum, boron, or silicon in borosilicate glassware and a silica based anion exchange columns (QMA) during handling of aqueous 18F for making a labeled product, comprises irradiating clear silanized borosilicate glass vials with [18O]H2O then analyzing with inductively coupled plasma atomic emission spectroscopy.

2. A method for detecting aluminum, boron, or silicon in borosilicate glassware and a silica based QMA during handling of aqueous 18F for making a labeled product, comprises preparing a K222/K2CO3 eluent mixture in 80/20 MeCN:H2O, storing 0.600-0.900 ml at room temperature in a 2-5 ml borosilicate glass vial, capped with a Teflon coated chlorobutyl stopper, and analyzing the content with inductively coupled plasma mass spectroscopy.

3. A method for detecting aluminum, boron, or silicon in borosilicate glassware and a silica based QMA during handling of aqueous for making a labeled product, comprises conditioning Sep-Pak® QMA light cartridges, originally on chloride form, with 10 ml 0.05 M K₂CO₃, washing with 20 ml WFI, drying under a stream of N₂ and storing at room temperature; thereafter 1.5 ml of WFI was forced through and measured directly by inductively coupled plasma atomic emission spectroscopy.

4. The method according to claim 1, wherein the labeled product is either an [18F] RGD peptide or anti-[¹⁸F]FACBC.

5. The method according to claim 2, wherein the labeled product is either an [18F] RGD peptide or anti-[¹⁸F]FACBC.

6. The method according to claim 3, wherein the labeled product is either an [18F] RGD peptide or anti-[¹⁸F]FACBC.

7. The method according to claim 2, wherein 0.825 ml of the K222/K2CO3 eluent mixture in 80/20 MeCN:H2O was stored at room temperature in a 3 ml borosilicate glass vial
